(19) United States
(12) Patent Application Publication
(10) Pub. No.: US 2013/0164275 A1
(43) Pub. Date: Jun. 27, 2013

(54) METHOD FOR TREATING ACIDOSIS IN RUMINANTS

(71) Applicants: Andrea Lacorte, Pisa (IT); Alessandro Lotto, Schio (IT)

(72) Inventors: Andrea Lacorte, Pisa (IT); Alessandro Lotto, Schio (IT)

(73) Assignee: PHARMACHEM LABORATORIES, INC., Kearny, NJ (US)

(21) Appl. No.: 13/504,409

(22) PCT Filed: Oct. 26, 2010
(86) PCT No.: PCT/US10/54071

§ 371 Date: Jan. 29, 2013

Related U.S. Application Data
(60) Provisional application No. 61/255,016, filed on Oct. 26, 2009.

(51) Int. Cl. A61K 38/47

(52) U.S. Cl. CPC A61K 38/47
USPC 424/94.61

(57) ABSTRACT

The present invention provides a method for treating acidosis in a ruminant. The method includes administering an effective amount of a naturally derived inhibitor of a carbohydrate degrading enzyme to the ruminant. Such enzymes include amylase and glucosidase. In addition, the carbohydrate degrading enzyme may be endogenous or exogenous to the ruminant.

### BACKGROUND OF THE INVENTION

Acidosis is a metabolic disease that negatively affects health and productivity in ruminants. For example, it can result in damage to the integrity of the gut wall and increased pathogenicity of gut bacteria and parasites.

In addition, acidosis in ruminants is associated with secondary conditions that can have a significant impact on livestock animal performance, i.e. reduction in the feed conversion to meat and/or milk. Milk quality can also suffer in association with acidosis because acidosis is associated with an increase in lactic acid production in the ruminant. The increased lactic acid production is correlated with a decreased production in volatile fatty acids, which negatively affects milk quality.

Other effects of acidosis in ruminants include laminitis, intermittent diarrhea, poor appetite, poor body condition, and abscesses without obvious causes. The animals may also have depressed immune system function, a high incidence of respiratory diseases, and reduced fertility rates.

According to Merck Veterinary Manual (4th edition), a current treatment of rumen acidosis involves emptying the contents of the rumen in the animal and replacing it with contents from a rumen of a healthy animal. Such a treatment is impractical, especially if a plurality of animals is affected.

Alternative treatments include administering synthetic compounds and solutions to the ruminant in order to correct the acid-base imbalance in the ruminant. Use of synthetic chemicals for treating ruminants has caused societal and environmental concerns since the ruminants produce and provide food products for human consumption. In addition, synthetic compounds may cause harmful side effects to the ruminant.

Treatments involving naturally-derived compounds, however, are considered to be more benign, less toxic, and have shorter half-lives than synthetic compounds. Accordingly, such treatments would be beneficial in the context of acidosis in ruminants.

Moreover, rumen acidosis and its related problems to the animals are estimated to cost the livestock industry more than $1 billion per annum due to lost performance. Accordingly, there is a general need for a safe and effective treatment for rumen acidosis.

### SUMMARY OF THE INVENTION

These and other objectives have been met by the present invention by providing a method for treating acidosis in a ruminant in need thereof. The method includes administering an effective amount of a naturally derived inhibitor of a carbohydrate degrading enzyme to the ruminant.

### DETAILED DESCRIPTION

The inventors have surprisingly discovered a method for treating acidosis in a ruminant in need thereof. The method includes administering an effective amount of a naturally derived inhibitor of a carbohydrate degrading enzyme to the ruminant.

Acidosis in ruminants is a metabolic disease that involves abnormally high acidic conditions in the gut. A major cause of acidosis is the consumption of a diet having a high content of readily fermentable carbohydrate and/or a diet that is low in roughage. Reduced pH in ruminants can also be caused by a decrease in the proportion of crude fiber in the diet.

Acidosis can be divided into several forms: acute, chronic, and sub-acute. Acute acidosis refers to a ruminant having a rumen pH between pH 4.0 and 5.0 with elevated ruminal lactate. Chronic acidosis refers to a ruminant having a rumen pH between 5.0 and 5.5 with normal levels of lactate of up to 5 mM. Subacute acidosis refers to a ruminant having a rumen pH value below 5.0. In some cases, subacute acidosis is also associated with high lactate levels.

Acidosis can be a systemic disease; however, forestomach acidosis affects ruminants especially. Forestomach acidosis refers to over-production of acid in the forestomach of a ruminant. The over-production of acid in the forestomach is typically caused by microorganisms in the forestomach.

A forestomach is the area of the stomach in which the major part of digestion in ruminants takes place, largely by fermentation. The forestomach, as used herein, refers to any part of the reticulum, rumen, and omasum, which are the first three compartments of a ruminant stomach.

Ruminants include, for example, cattle, goats, sheep, giraffes, bison, yaks, water buffalo, deer, camels, alpacas, llamas, wildebeest, antelope, pronghorn, and nilgai. Preferably, the ruminant is bovine.

A ruminant in need thereof is, for example, any ruminant that has an abnormally high acidic condition in its forestomach. In one embodiment, such a ruminant has a rumen pH that is preferably less than 5.5 at 12 hours after digestion of carbohydrates, or more preferably less than pH 4.5 by 12 hours after digestion of carbohydrates. Such ruminants may also include those that have consumed, for example, a high content of readily fermentable carbohydrates, a low quantity of roughage, and/or a decrease in the proportion of crude fiber in the diet. Additional examples of ruminants in need thereof include, but are not limited to, ruminants that suffer from damage to the integrity of the gut wall, or from increased pathogenicity of gut bacteria and parasites. Other examples include ruminants that have a reduction in the feed conversion to meat and/or milk, and those experiencing laminitis, intermittent diarrhea, poor appetite, poor body condition, and abscesses without obvious causes.

According to the invention, an effective amount of a naturally derived inhibitor of a carbohydrate degrading enzyme is administered to the ruminant.

A carbohydrate degrading enzyme refers to an enzyme that is responsible for breaking down carbohydrates in a ruminant. Such enzymes include amylase and glucosidase.

Carbohydrates are composed of one or more sugar units linked together through glycosidic bonds. Complex carbohydrates, such as starch, are relatively large molecules containing numerous repeating units formed into a multi-branched chain structure. Monosaccharides and disaccharides, on the other hand, are simple carbohydrates that contain single and double sugar units, respectively.

Complex carbohydrates are typically degraded in a ruminant by carbohydrate degrading enzymes that break down carbohydrates into simpler units, e.g., monosaccharides. The ruminant then absorbs these simpler units into tissue, such as intestinal tissue.

A variety of digestive enzymes works in a stepwise manner to break down the complex carbohydrates into the absorbable units. Examples of carbohydrate degrading enzymes include amylase and glucosidase, preferably, alpha-amylase and alpha-glucosidase.

Amylase is an enzyme that is known to break down large carbohydrates into smaller units of oligo- and disaccharides. Alpha-amylase is an enzyme that functions to break the alpha-1,4-glycosidic linkages present in starch. The enzyme breaks down the complex starch molecule into smaller units, such as disaccharides, that can be further digested by other enzymes, such as alpha-glucosidase.

Glucosidase can break down the disaccharides, maltose and sucrose, into their respective monosaccharide units, which can then be absorbed by the body. Alpha-glucosidase is an enzyme that breaks disaccharides into their respective monosaccharide units.

In one embodiment, the carbohydrate degrading enzyme may be endogenous or exogenous to the ruminant. An endogenous enzyme refers to an enzyme that is naturally produced inside the ruminant, and is not exogenous. For example, amylase can be naturally produced by a ruminant in its saliva or by its pancreas. Glucosidase can be found in the intestinal tract, e.g., produced by the ruminant's pancreas, and in saliva of the ruminant.

An exogenous enzyme refers to an enzyme that is introduced into or produced outside a cell or system of the ruminant. An exogenous enzyme includes enzymes that are produced by bacteria within the ruminant.

Preferably, the exogenous enzyme is produced by bacteria that are within the forestomach of the ruminant. For example, amylase can be produced by a wide variety of microorganisms, some of which may are considered species of microflora in a ruminant forestomach, including Bacillus and Aspergillus species, namely, Bacillus licheniformis, Bacillus amyloliquefaciens, Bacillus subtilis, and Bacillus stearothermophilus, and including Streptococcus ruminantium, Streptococcus bovis, Propionibacterium ruminicola, Lactobacillus vitulinus, Megasphaera elsdenii, Ruminobacter amylophilus, Succinimonas amylolytica. In addition, microflora of a ruminant may include other species of Bifidobacterium, Butyrivibrio, Eubacterium, Lactobacillus, Mitsuokella, Prevotella, Ruminobacter, Selenomonas, Streptococcus, Succinimonas, and Succinivibrio. Glucosidase can also be produced by a wide variety of microorganisms, such as those listed above.

An inhibitor of a carbohydrate degrading enzyme includes glycoproteins that inhibit the enzyme responsible for breaking down carbohydrates in ruminants. Exemplary inhibitors include amylase inhibitors and glucosidase inhibitors. More preferable inhibitors include alpha-amylase inhibitors and alpha-glucosidase inhibitors.

Exemplary alpha-amylase inhibitors prevent the alpha-amylase enzyme from hydrolyzing the alpha-1 ,4-glycosidic bond, and therefore prevent the breakdown of starch. A wide variety of alpha-amylase inhibitors are known, and any suitable inhibitor can be used in the methods of the present invention. Examples of suitable alpha-amylase inhibitors include, but are not limited to, an inhibitor extracted from wheat (see U.S. Pat. No. 3,950,319 to Schmidt et al.), amylostatin-A (see U.S. Pat. No. 4,010,258 to Murao), and phaseolamin.

Exemplary alpha-glucosidase inhibitors prevent the enzyme from performing this function. A wide variety of alpha-glucosidase inhibitors are known and any suitable inhibitor can be used in the compositions and methods of the present invention. Examples of suitable alpha-glucosidase inhibitors include, but are not limited to, voglibose (see U.S. Pat. No. 6,200,958 to Odaka et al.), acarbose (see U.S. Pat. No. 5,643,874 to Bremer et al.), and touchi extract.

The inhibitor of the invention is preferably derived from a bean. For example, touchi is derived from soybeans. Touchi is prepared by first steaming and then fermenting soybeans with Aspergillus species bacteria. Typically, touchi is used for food purposes in a paste form. The touchi useful in the present invention is an extract of touchi.

Another suitable bean for use in the invention belongs to the Phaseolus vulgaris family which includes, for example, kidney beans. The beans are typically small, intact beans. Preferably, the inhibitor is derived from white kidney beans. In another preferred embodiment, the beans are not genetically modified beans.

The amylase inhibitor from white kidney beans is sometimes referred to as “phaseolamin.” Phaseolamin is an extract of the white kidney bean (Phaseolus vulgaris). The extract is water-soluble and rich in protein content.

The amylase inhibitor of the invention is superior to other amylase inhibitors because it has a higher degree of purity than amylase inhibitors obtained by conventional extraction methods, i.e. heat and chemical. Due to the high degree of purity, the amylase inhibitor exhibits improved stability and potency in vitro and in vivo over amylase inhibitors of the prior art. Likewise, the glucosidase inhibitor of the invention is superior to other glucosidase inhibitors due to its higher degree of purity.

The naturally-derived amylase inhibitor and glucosidase inhibitor of the present invention remain stable at elevated temperatures, such as, for example, 120-200° F. Such heat stability allows the amylase inhibitor and glucosidase inhibitor to be utilized in, for example, food products that are cooked or processed, without losing the beneficial, starch-blocking effects of the inhibitors.

The naturally-derived amylase inhibitor and glucosidase inhibitor also remain intact at extreme pH values. For example, the stomach can have a pH of approximately 1-2. The naturally-derived amylase inhibitor and glucosidase inhibitor of the invention remain mainly intact under such pH conditions.

In addition, the naturally-derived amylase inhibitor and glucosidase inhibitor are more potent than the amylase or glucosidase inhibitors derived from conventional heat/solvent methods. Not being bound by theory, it is proposed that by avoiding the use of chemical solvents, the important tertiary structure of the amylase and/or glucosidase inhibitor are not disrupted.

Preferably, the inhibitor of a carbohydrate degrading enzyme is administered systemically. Systemic administration can be enteral or parenteral. Enteral administration is preferred. For example, the naturally-derived amylase inhibitor and glucosidase inhibitor can be easily administered orally. Liquid or solid (e.g., feed paste, pill, gel, tablets, gelatin capsules) formulations can be employed. The formulations can include pharmaceutically acceptable excipients, adjuvants, diluents, or carriers.

In one embodiment, the naturally derived inhibitor of a carbohydrate degrading enzyme is administered to the ruminant in total mixed ration systems (TMR). For example, the total mixed ration (TMR) ruminant feed may provide a total diet for the ruminant, except for salt and water. The TMR may provide all the daily nutrients required for a particular type, size, age, and/or activity of ruminant.

In an alternative embodiment, the naturally derived inhibitor of a carbohydrate degrading enzyme is administered to the ruminant in a concentrate feed, mineral feed, pelleted feed, industrial feed premix, commercial forage, or concentrate mix.

In accordance with the present invention, an effective amount of the amylase inhibitor and/or glucosidase inhibitor is any amount known to those skilled in the art to effectively inhibit the breakdown of dietary starch. Preferably, an effective amount is administered to a ruminant just prior to, during, or shortly after, consuming a starch-containing meal. For example, a typical pre-meal dosage of the amylase inhibitor is approximately 500 mg to 1,500 mg.

In one preferred embodiment, the naturally-derived amylase inhibitor and/or glucosidase inhibitor is administered to the ruminant at a rate between 5 and 100 mg/kg of live total body weight of the ruminant. More preferably, the inhibitor is administered to the ruminant at a rate between 7 and 100 mg/kg of live total body weight of the ruminant.

### EXAMPLES

A gas production test using single maize, wheat, and liquid pure starch was run on rumen bacteria. Another gas production test was run using real corn and real wheat (without purified starch). A desy test (using artificial rumen) will be performed in which the Phaseolus extract will be in contact with the total mixed ration (TMR) containing the starch sources.

The gas method allows study of effects of the feed on digestibility of feed fractions and rumen microbial activity. An in vitro rumen gas production technique can be used to evaluate commercial total mixed rations (TMR) for total gas production. Methods of rumen gas production technique and variations thereof are known in the art. See, for example, DePeters, et al, 2003. “In vitro rumen gas production as a method to compare fermentation characteristics of steam-flaked corn.” Anim. Feed Sci. Technol. 105, 109-122; Getachew, et al., 2002a. “Tropical browses: contents of phenolic compounds, in vitro gas production and stoichiometric relationship between short chain fatty acid and in vitro gas production.” J. Agric. Sci. 139, 341-352; Getachew, et al., 2002b. “Laboratory variation of 24 h in vitro gas production and estimated metabolizable energy values of ruminant feeds.” Anim. Feed Sci. Technol. 102, 171-182; and Menke, et al., 1979. “The estimation of the digestibility and metabolisable energy content of ruminant feedingstuffs from the gas production when they are incubated with rumen liquor.” J. Agric. Sci. 93, 217-222.

What is claimed:

1. A method for treating acidosis in a ruminant in need thereof comprising administering an effective amount of a naturally derived inhibitor of a carbohydrate degrading enzyme to the ruminant.

2. The method according to claim 1 wherein the inhibitor of a carbohydrate degrading enzyme is derived from a bean.

3. The method according to claim 2 wherein the bean is Phaseolus vulgaris.

4. The method according to claim 2 wherein the carbohydrate-degrading enzyme is endogenous.

5. The method according to claim 4 wherein the enzyme is a pancreatic enzyme.

6. The method according to claim 4 wherein the enzyme is amylase.

7. The method according to claim 4 wherein the enzyme is glucosidase.

8. The method according to claim 2 wherein the carbohydrate degrading enzyme is exogenous.

9. The method according to claim 8 wherein the enzyme is produced by bacteria.

10. The method according to claim 9 wherein the bacteria are carbohydrate-fermenting bacteria in a forestomach of the ruminant.

11. The method according to claim 8 wherein the enzyme is amylase.

12. The method according to claim 8 wherein the enzyme is glucosidase.

13. The method according to claim 1 wherein the acidosis is chronic rumen acidosis.

14. The method according to claim 1 wherein the acidosis is acute rumen acidosis.

15. The method according to claim 1 wherein the acidosis is subacute rumen acidosis.

16. The method according to claim 1 wherein the acidosis is forestomach acidosis.

17. The method of claim 16 wherein the forestomach acidosis is acidosis of the rumen, reticulum, or omasum of the ruminant.

18. The method according to claim 1 wherein the administration comprises a feed paste, pill, gel, gel cap, or tablet.

19. The method according to claim 1 wherein the ruminant is bovine.
